(19) United States
(12) Patent Application Publication
(10) Pub. No.: US 2005/0013924 A1
(43) Pub. Date: Jan. 20, 2005

(54) Method for formulating healthcare products with enhanced stability

(76) Inventors: Ramaswamy Murari, Hillsborough, NJ (US); Ashok Katdare, Highland Park, IL (US); Suggy S. Chrai, Cranbury, NJ (US); Troy M. Harmon, Lansdale, PA (US)

Correspondence Address: JOHN W. RYAN;C/O DECHERT LLP, PRINCETON PIKE CORPORATION CENTER, P.O. BOX 5218, PRINCETON, NJ, 08543-5218, US

(21) Appl. No.: 10/332,255

(22) PCT Filed: Jul. 6, 2001
(86) PCT No.: PCT/US01/21418

Related U.S. Application Data
(60) Provisional application No. 60/216,205, filed on Jul. 6, 2000.

(51) Int. Cl. A61L 2/00

(52) U.S. Cl. 427/2.1

(57) ABSTRACT

A method is disclosed for formulating healthcare products, including solid pharmaceutical compositions, while avoiding instability caused ba interaction of the active ingredient with excipients. The method comprises the steps of: (a) selecting an active ingredient that loses stability or potency upon interaction with pharmaceutical excipients; and (b) depositing the active ingredient, preferably electrostatically, as a dry powder substantially free of excipients, onto a pharmaceutically acceptable polymer substrate.

The present invention relates to improved healthcare product formulations, including solid pharmaceutical dosages. This application is related to co-pending application U.S. Ser. No. ______, “Improved Thyroid Hormone Formulations,” filed contemporaneously with the present application and assigned to the same assignee, the disclosure of which is hereby incorporated by reference in its entirety.

### BACKGROUND OF THE INVENTION

Modern healthcare product manufacturing processes have become increasingly sophisticated. For example, newer techniques for manufacturing pharmaceutical dosages involve higher pressures, dry processing, and the like. In addition, new compounds are being formulated, including, but not limited to, proteins, peptides, enzymes, hormones, nucleic acids and derivatives thereof (collectively, “drugs of biological origin”). Further, more complex formulations are being manufactured in an attempt not only to improve bioavailability and extend shelf-life, but also to reduce toxicity and to enable site-specific drug delivery.

Solid pharmaceutical dosages traditionally have included capsules, tablets and other unit dosage forms, each form containing a pharmaceutically or biologically active ingredient and at least one additional “excipient” ingredient. The excipient, which is intended to be a therapeutically inert and non-toxic carrier, may function, for example, as a diluent, binder, lubricant, disintegrant, stabilizer, buffer or preservative.

Various active pharmaceutical ingredients, when admixed with excipients, have exhibited problems relating to chemical stability. Some prominent examples have included lansoprazole, molsidomime, topotecan, levothyroxine, moexipril, oxprenolol, Astra FLA 336, nifedipine, prednisone, nitroglycerine, heparin, as well as the above-identified drugs of biological origin.

In fact, some of the most widely used “inert” excipients may be quite reactive in their own right. Drugs can interact with excipients via a number of mechanisms, resulting in chemical instability and degradation. The examples are numerous: Any easily hydrolyzable drug should not be mixed with a hydrated excipient if the water of crystallization could be released by the formulating process. If the active ingredient has a primary amine function, the use of mono- or di-saccharide excipients may lead to amine-aldehyde and amine-acetal reactions. If the active ingredient is an ester or lactone, the use of any excipient that might create a basic environment could lead to ester-base hydrolysis. Any compound containing an aldehyde moiety should not be mixed with amine type excipients, in order to avoid aldehyde-amine reactions. Finally, the formation of hydrogen bonds, such as those between carbonyl and silanol groups, may destabilize a drug.

Moreover, even where an excipient itself would be inert to the active component of a formulation, the excipient still may contain some impurities, such as unreacted metals or residual solvents, whose origin lies in the processing of the excipient. These impurities can then react and/or degrade the drug, and reduce its activity. For example, ferric iron catalyzes the oxidation of drugs such as hydrocortisone. Thus, clays containing adsorbed ferric iron should be avoided in formulating drugs prone to such oxidative degradation. As another example, aldehydes and peroxides may be present as reactive impurities in polyethylene glycol (PEG).

Further, although the manufacture of most solid dosage forms requires compression forces at some stage, relatively little is known about the interactions that can occur between ingredients during, and just after, such compression. There is evidence that polymorphic transformation of certain active ingredients or excipients, as well as solid-solid interactions between certain active ingredients and excipients, can arise upon mechanical handling. The amount of pressure and the duration for which it is applied, as well as the number of compressions to which a formulation is subjected, are factors which may contribute to the extent of ingredient modification and interaction. For example, lubricants, such as magnesium stearate, have been found to have a deleterious effect on solid state stability. Compression forces can liquefy low-melting lubricants, which then dissolve the drug, changing its properties.

Various approaches have been suggested in order to prevent such harmful interaction between active ingredients and excipients. One possibility is to embark on a lengthy, and possibly fruitless, search for alternate excipients. Another possible solution is to separate the interacting ingredients by coating either the active ingredient or the excipient, or by preparing some kind of partitioned dosage form. However, this approach adds complexity and expense to the formulation process.

Therefore, it would be desirable to provide a reliable, as well as less complex and less expensive, method for formulating a pharmaceutical composition while avoiding instability caused by interaction of the active ingredient with excipients.

### SUMMARY OF THE INVENTION

In accordance with the teachings of the present invention, a method is provided for formulating healthcare products, including solid pharmaceutical dosages, with enhanced stability, which overcomes the disadvantages of the approaches suggested in the prior art. The method of formulating a healthcare product, including a pharmaceutical composition, while avoiding instability caused by interaction of the active ingredient with excipients, comprises the steps of.
(a) selecting an active ingredient that loses stability or potency upon interaction with pharmaceutical excipients; and
(b) depositing the active ingredient, preferably electrostatically, as a dry powder substantially free of excipients, onto a pharmaceutically acceptable polymer substrate.

It is accordingly an object of the present invention to provide a method for formulating healthcare products, including solid pharmaceutical dosages, with enhanced stability, without regard to the nature of any undesirable interaction between the active ingredient and certain excipients.

### DETAILED DESCRIPTION OF THE INVENTION

In one embodiment of the present invention, drugs or pharmaceutical agents can be formulated into a suitable dosage form with increased stability by depositing the pharmaceutical active agent as a pure ingredient onto a substrate in the absence of excipients and then processing into an appropriate dosage form therefrom. In a preferred embodiment, a pharmaceutical active agent, that has been found to have stability problems when admixed with excipients, is deposited, as a dry powder substantially free of excipients, onto the substrate by an electrostatic deposition process.

In the electrostatic deposition process, a cloud or stream of charged particles of the active ingredient is exposed to, or directed towards, a substrate, at the surface of which substrate a pattern of opposite charges has been established. In this fashion, a measured dosage of the active ingredient can be adhered to a substrate without the need for additional carriers, binders or the like. Thus, in a preferred embodiment, pharmaceutically active agents, that normally are unstable when admixed with excipients and/or subjected to normal mechanical processing conditions involved in the manufacture of traditional solid dosage forms, are stable when incorporated into a final dosage form using a process of the invention, involving electrostatic deposition.

Suitable means of electrostatic deposition are described in, for example, U.S. Pat. Nos. 5,714,007, 5,846,595 and 6,074,688, the disclosures of which are incorporated by reference herein in their entireties.

Active pharmaceutical ingredients that would benefit from the enhanced-stability formulation method of the present invention include, but are not limited to, lansoprazole, molsidomime, topotecan, moexipril, oxprenolol, Astra FLA 336, nifedipine, steroids (e.g., prednisone), nitroglycerine, heparin, and drugs of biological origin. It should be understood that, in addition to the active ingredients included in this list, any other suitable active pharmaceutical ingredient, which demonstrates instability or loss of potency when compressed or when admixed with various excipients, can easily be identified and selected by those of ordinary skill in the art, by routine testing.

The preferred deposition substrate is a “pharmaceutically acceptable” polymer; that is, one that may be introduced safely into the human or animal body, for example, taken orally and digested. Ideally, the polymer has received regulatory approval and is of GRAS (“Generally Regarded As Safe”) status. The substrate polymer, preferably in the form of a film, may either dissolve or otherwise disintegrate subsequent to introduction into the body, for example, subsequent to or upon ingestion, or the polymer may be substantially inert and pass through the body, provided that the dosage form opens or otherwise releases the pharmaceutical substance from the deposit into the patient's body. Suitable materials may include, for example, polymers and copolymers of polyvinyl alcohol, polyvinyl pyrrolidinone, polysaccharide polymers, acrylate polymers, methacrylate polymers, phthalate polymers, polyvinyl acetate, methyl cellulose, carboxymethylcellulose, hydroxyethylcellulose, hydroxypropylcellulose, hydroxypropylmethylcellulose, ethyl cellulose, Eudragits (that is, polymers and copolymers containing methacrylic acid), starch-based polymers, gelatin and the like.

Preferred dosage forms, as well as additional useful substrate polymers, are disclosed in published international patent application number WO 99/63972, the disclosure of which hereby is incorporated by reference herein in its entirety. For example, a cover film may be applied to encapsulate the electrostatically deposited active ingredient, and the resulting stable “core” may be further processed into dosage forms resembling conventional tablets, capsules, caplets and the like or processed into non-conventional wafers or stamp-like presentations. The preferred dosage forms may be suitable for oral, transdermal or buccal dosing of appropriate drugs. The method of the invention provides satisfactorily small dosages as may be required, for example, for insulin and its derivatives, heparin and other orally absorbed drugs.

### EXAMPLES

### Example 1

The compatibility of various conventional polymer films with levothyroxine sodium was evaluated. The goal was to select a suitable polymer film to maximize the stability of levothyroxine sodium for electrostatic deposition, and to develop a dosage form using selected polymer films.

Each sample was prepared by depositing levothyroxine sodium on a polymer substrate, in a drug-to-film ratio of approximately 1:14. Samples were stored in individual amber vials with Teflon-lined screw cap closures at 25° C. with 60% Relative Humidity and at 40° C. with 75% Relative Humidity (“RH”). As a control, levothyroxine sodium drug substance was stored, without any deposition substrate, in closed amber vials under the same conditions as the samples. Samples were analyzed at 4 or 6 weeks for the presence of degradants (and loss of active ingredient) by means of a stability-indicating High Performance Liquid Chromatography method.

The following polymers and copolymers were evaluated:
1. Substrate 1527-79-1: 50% Hydroxypropylmethylcellulose (“HPMC”)+50% Hydroxypropylcellulose (“HPC”)
2. Substrate 1577-7-1: 60% Ethyl cellulose (“EC”)+5% HPMC+35% Triethyl citrate (“TEC”)
3. Substrate 1577-7-3: 60% EC+5% HPC+35% TEC
4. Substrate 1577-6-3: 66% Cellulose acetate phthalate (“CAP”)+5% HPMC+25% TEC+4% Polysorbate 80
5. Substrate 1577-6-5: 66% CAP+5% HPC+25% TEC+4% Polysorbate 80
6. Substrate 1527-69-1: 45% HPMC+45% HPC+10% Polyethylene Glycol 400 (“PEG”)
7. Substrate 1527-84-1: 100% BPC
8. Substrate 1501-56-3: 100% HPMC

The following is a summary of the stability of certain formulations (that is, the percentage of active ingredient remaining) at four weeks:

| | 25° C./60% RH | 40° C./75% RH |
|---|---|---|
| 1527-69-1 | 98.8% | 98.8% |
| 1527-79-1 | 98.8% | 98.7% |
| 1577-7-1 | — | 99.0% |
| 1577-7-3 | 99.1% | 99.0% |
| 1577-6-3 | 94.3% | 60.8% |
| 1577-6-5 | 93.8% | 51.3% |

The stability of certain formulations at six weeks was as follows:

| | 25° C./60% RH | 40° C./75% RH |
|---|---|---|
| 1527-79-1 | 98.7% | 98.8% |
| 1527-84-1 | 98.7% | 98.7% |
| 1501-56-3 | 98.6% | 98.7% |
| 1577-7-1 | 99.0% | 98.7% |
| 1577-7-3 | 98.7% | 98.0% |
| 1527-69-1 | 98.3% | 95.3% |

The results indicate that certain polymers were associated with an undesirable loss of active ingredient. However, five of the eight polymer film formulations were associated with a loss of no more than 2% of the active ingredient under stress conditions. Thus, it is apparent that polymers having a high degree of compatibility with an active ingredient (that is, which result in negligible loss of the active ingredient) can be identified readily from the routine screening of polymers that are conventional for pharmaceutical use.

### Example 2

The compatibility of three polymer films with ondansetron was evaluated. The goal was to select a suitable polymer film to maximize the stability of ondansetron for electrostatic deposition, and to develop a dosage form using selected polymer films.

Each sample was prepared by depositing a quantity of ondansetron on a polymer substrate followed by folding of the film. Each sample was stored in a high-density polyethylene (HDPE) bottle with polypropylene (PP) screw cap at 25° C. with 60% Relative Humidity and at 40° C. with 75% Relative Humidity (“RH”). As a control, ondansetron drug substance was stored, without any substrate, in HDPE bottles with PP screw caps under same conditions as the samples. Samples were analyzed at 2 or 4 weeks for the presence of degradants (and loss of active ingredient) by means of a stability-indicating High Performance Liquid Chromatography method.

The following polymers and copolymers were evaluated:
1. Substrate 990210: Purity Gum, Sorbitol and Pectin
2. Substrate 990193: 45% Hydroxypropylmethylcellulose (“HPMC”)+45% Hydroxypropylcellulose (“HPC”)+10% Polyethylene Glycol 400 (“PEG”)
3. Substrate 990077: Hydroxypropylmethylcellulose (“HPMC”)

The following is a summary of the stability of certain formulations (that is, the percentage of active ingredient remaining) at two weeks:

| | 25° C./60% RH | 40° C./75% RH |
|---|---|---|
| 990210 | 99.8% | 99.9% |
| 990193 | 99.9% | 99.9% |
| 990077 | 99.9% | 99.9% |

The stability of certain formulations at four weeks was as follows:

| | 25° C./60% RH | 40° C./75% RH |
|---|---|---|
| 990210 | 99.9% | 99.9% |
| 990193 | 100.0% | 99.9% |
| 990077 | 100.0% | 100.0% |

The results indicate that all the polymer film formulations in the study were associated with the loss of no more than 1% of the active ingredient under stress conditions, indicating a high degree of compatibility with the active ingredient.

Although the present invention has been described with particular reference to certain preferred embodiments thereof, variations and modifications of the present invention can be effected within the spirit and scope of the following claims.

1. A method of formulating a healthcare product while avoiding instability caused by interaction of the active ingredient with excipients, the method comprising:
(a) selecting an active ingredient that loses stability or potency upon interaction with pharmaceutical excipients; and
(b) depositing the active ingredient, as a dry powder substantially free of excipients, onto a pharmaceutically acceptable polymer substrate.

2. The method of claim 1, wherein the depositing is performed electrostatically.

3. The method of claim 2, wherein the healthcare product is a solid pharmaceutical dosage.

4. The method of claim 1, wherein the polymer has received regulatory approval and is of GRAS status.

5. The method of claim 4, wherein the polymer is selected from the group consisting of polyvinyl alcohol, polyvinyl pyrrolidinone, polysaccharide polymers, acrylate polymers, methacrylate polymers, phthalate polymers, polyvinyl acetate, methyl cellulose, carboxymethylcellulose, hydroxyethylcellulose, hydroxypropylcellulose, hydroxypropylmethylcellulose, ethyl cellulose, Eudragits, starch-based polymers, gelatin, and combinations thereof.

6. The method of claim 3, wherein the active ingredient is selected from the group consisting of lansoprazole, molsidomime, topotecan, moexipril, oxprenolol, Astra FLA 336, nifedipine, steroids, nitroglycerine, heparin, insulin and drugs of biological origin.

7. The method of claim 3, further comprising:
(a) applying a cover film to encapsulate the electrostatically deposited active ingredient, so as to form a stable core; and
(b) further processing the stable core into a dosage form resembling a tablet, capsule, caplet, wafer or stamp-like presentation.

8. An improved solid pharmaceutical dosage formulation, comprising a therapeutic amount of an active pharmaceutical ingredient, deposited on a pharmaceutically acceptable polymer substrate as a dry powder substantially free of excipients.

9. The formulation of claim 8, wherein the active ingredient is deposited electrostatically.
